(19) United States
(12) Patent Application Publication
(10) Pub. No.: US 2019/0264174 A1
(43) Pub. Date: Aug. 29, 2019

(54) METHOD OF CULTIVATION OF HUMAN SALIVARY GLAND CELLS

(71) Applicant: PIROGOV RUSSIAN NATIONAL RESEARCH MEDICAL UNIVERSITY (RNRMU), Moscow (RU)

(72) Inventors: Olga Sergeevna PETRAKOVA, Balashikha (RU); Mikhail Aleksandrovich BORISOV, Moscow (RU); Ekaterina Andreevna VOROTELYAK, Moscow (RU); Inessa Givievna GVAZAVA, Moscow (RU); Olga Sergeevna ROGOVAYA, Reutov (RU); Andrey Valentinovich VASILIEV, Moscow (RU)

(73) Assignee: PIROGOV RUSSIAN NATIONAL RESEARCH MEDICAL UNIVERSITY (RNRMU), Moscow (RU)

(21) Appl. No.: 16/339,548

(22) PCT Filed: Oct. 3, 2017
(86) PCT No.: PCT/RU2017/000736

§ 371 Date: Apr. 4, 2019

(30) Foreign Application Priority Data
Oct. 6, 2016 (RU) 2016139283

(51) Int. Cl. C12N 5/071

(52) U.S. Cl. CPC C12N 5/0633; C12N 2500/25; C12N 2500/84; C12N 2500/32; C12N 2523/00; C12N 2501/11

(57) ABSTRACT

The present invention is intended to increase the number of human salivary gland cell passages, maintain their undifferential condition and high proliferative potential during cultivation. The culture method of human salivary gland epithelial progenitor cells comprising: (a) obtaining human salivary gland epithelial progenitor cells from recipient organism; (b) cell transfer into PCT Epidermal Keratinocyte Medium and cultivation in culture flasks ensuring cell adhesion at 37° C. with addition of 5% CO₂ and medium change every 2-4 days until monolayer is reached; (c) cell passage at 1:3-1:5 dilution ratio, including cell removal from the culture flask surface using EDTA trypsin solution and transfer into the new culture flasks; (d) further cell cultivation as defined in claim (b) with in-process medium change every 2-4 days and passaging until monolayer is reached, as defined in claim (c) at a maximum dilution ratio of 1:2-1:3, where the first medium change after each passage shall be provided within 8-24 hours.

### FIELD OF THE INVENTION

The invention relates to cellular biology. In particular, the invention relates to human cell culture.

### BACKGROUND OF THE INVENTION

In certain cases, cultivation of human cells intended to increase biomass of the target cell culture appears to be a challenging task. Human salivary gland epithelial progenitor cell culture causes significant problems. There are known methods for obtaining salivary gland cells of mammals, for instance, a rat [Okumura K. et al., Hepatology. 2003. 38. 104-113], a mouse [Hisatomi Y. et. al., Hepatology. 2004. 39(3). 667-675; Ikeura, K. et al., Plos One. 2016, e0147407], a pig [Matsumoto S., et al., Cloning and Stem Cells. 2007. 9. 176-190]. However, the methods suitable for animal cells are not applicable for long-term cultivation of human salivary gland epithelial progenitor cells. It is found that these human cells cultivated in vitro are prone to spontaneous differentiation resulting in a substantial cell increase, granular cytoplasm and proliferation potency loss. Thus, long-term cultivation of salivary gland epithelial cells becomes impossible [Sabatini, L. M., et al., In Vitro Cell Dev. Biol. 1991 27A 939-948]. For instance, mouse salivary gland epithelial cells are easy cultivated with more than 90 passages on DMEM/F12 1:1 medium, with addition of 10% fetal calf serum, whereas in contrast, human cells on this medium are not capable of being continuously cultivated.

So, those long-term culture methods of human salivary gland epithelial cells are in demand which ensure maintaining undifferentiated cell state for at least 15 passages. When using such methods, it is possible to provide considerable cell propagation (more than 200 million cells from a small 0.5-3 cm³ bioptic sample).

Some research works describe culture methods of human salivary gland mesenchyme cells. So, the research work [Jeong J. et al., Exp. Mol. Med. 2013. 45:e58] describes the culture method of human parotid or submandibular salivary gland cells: the cells were isolated by collagenase, seeded into I type collagen coated culture flasks and cultivated on DMEM/F12 1:1 medium with addition of 10% fetal calf serum, 1% penicillin/streptomycin, 1% insulin-transferrin-selenite, 10 mM nicotinamide, 100 nM dexamethasone, 1 mM β-mercaptoethanol, 20 ng/ml EGF (epidermal growth factor), 20 ng/ml HGF (hepatocyte growth factor), 20 ng/ml oncostatin M, 1000 U/ml LIF (leukemia inhibitory factor). These cells expressed mesenchyme cell markers, such as CD44, CD49f, CD90 and CD105. In addition, they had adipogenic, osteogenic and chondrogenic differentiation potency and went through 10 passages.

The research work [Schwarz S., Rotter N. Methods Mol. Biol. 2012. 879. 403-442] describes plastic cultivation on DMEM/F12 1:1 medium with addition of 10% fetal calf serum and 1% penicillin/streptomycin. The cells had typical fibroblast-like morphology, expressed CD29, CD44, CD73, CD90, CD105 and had adipogenic, chondrogenic and osteogenic differentiation potency.

Thus, when applying the culture methods described in Jeong J. and coauthors, and Schwarz S., Rotter N. [Jeong J. et al., Exp. Mol. Med. 2013. 45:e58; Schwarz S., Rotter N. Methods Mol. Biol. 2012. 879. 403-442], the media are suitable for mesenchyme cell culture, and the growth of human salivary gland epithelial cells is not observed.

The culture method of human parotid salivary gland immortalized epithelial cell lines [U.S. Pat. No. 5,462,870 dated 31 Oct. 1995 “Human diploid salivary gland epithelial cell lines”]. The lines are diploid cells derived from normal parotid salivary gland of a man (HPAM1) and a woman (HPAF1). The cells have continuous serum-free cultivation potency on KBM (Keratinocyte Basal Medium) with addition of 5 μg/ml insulin, 0.5 μg/ml hydrocortisone, 10 ng/ml EGF (epidermal growth factor), 25 μg/mI bovine pituitary extract, 100 IU/ml penicillin and 100 μg/ml streptomycin. Before passaging the cells are to be washed with HBSS, removed from plastic using 0.125% trypsin and incubated in KBM containing 5% fetal calf serum for trypsin inhibition. Then the cells are pelleted and resuspended in the described above serum-free growth medium. Cell lines have cryogenic freezing and long-term cryogenic storage abilities. The cells express α-keratine, amylase and proline-rich protein (PRP).

Immortalized cell lines can be used for study of different processes: biochemical and molecular mechanisms of growth and differentiation, cell cooperation, oncotransformation, cytotoxicity of various substances, cytokine expression, etc. However, such cells are not intended for medical applications during cellular therapy for safety reasons.

There is known method for obtaining human salivary gland stem cells [WO 2014092575 dated 19 Jun. 2014 “Means and methods for obtaining salivary gland stem cells and use thereof”]. This method relates to obtaining human salivary gland stem cells (mainly submandibular or parotid salivary glands), their transplantation for cellular therapy, including xerostomia. Salivary gland bioptic sample is mechanically divided into 1-5 mm³ fragments, then treated with collagenase and hyaluronidase, infiltrated (pore size 100 μm) in order to obtain monocellular suspension. Stem cells with maximum potential and expressing EpCAM, c-Kit, CD49f, CD29, CD133 and CD24 markers can be sorted out. Then suspended cultivation is provided in order to obtain salispheres—3D circular-type structures. The following additives are used for the culture medium: antibiotic, glutamine, 20 ng/ml EGF (fibroblast growth factor), 10 μg/ml insulin, 1 μM dexamethasone and N-2. Salispheres obtained from a single cell can be further cultivated after treatment with trypsin. After resuspending and obtaining multi-cellular suspension, the cells on the culture medium with the above mentioned additives are transplanted into 3D matrix for the second generating of salispheres. Proteins of basal membrane components, such as IV type collagens and laminins, for instance, matrigel, are used as 3D matrix. Salispheres can be isolated from the matrix using dispase, and then treated with trypsin to provide the following salisphere generation. Salispheres reach 50-80 μm in size within 10 days. Salispheres can be differentiated in order to obtain organoids—analogues of organism structures. Single salispheres are transplanted into matrigel containing I type collagen in the above mentioned medium with addition of gamma secretase inhibitor or 10% fetal calf serum. Salispheres are being differentiated for about 1 month to obtain organoid. This method has the following disadvantages: 3D cell culture is labour-consuming and technically complex process. It is known that in 3D structures a number of cells die and other cells differentiate and lose their proliferative potential [Lin R. Z., Chang H. Y. Biotechnol. J. 2008. 3(9-10). 1172-1184]. For this reason, when passaging the cell mass gain appears to be insignificant. To provide differentiation this method offers cultivation of single salispheres what is technically challenging and expensive. The process of organoid obtaining takes about a month, and so, the whole culture and differentiation process is time-consuming. Thus, it is impossible to increase the cell bulk (more than 200 million cells) when using this method. In addition, matrigel is used as a matrix during cultivation and differentiation processes what fails to meet biological safety requirements considering that matrigel is obtained from tumor material.

The work is known that describes obtaining the cells from human minor salivary glands [Jang S. I. et al., J. Dent. Res. 2015. 94(2). 304-311]. The cells were cultivated in collagen coated culture flasks on Keratinocyte growth medium (KGM) with addition of bovine pituitary extract, EGF, insulin, hydrocortisone, gentamycine, epinephrine and transferrin. The cells had typical epithelial morphology, small sizes and potential for 10 passages. The cells expressed cytokeratins 5 and 18 and nanog. Thus, the disadvantages of the current method are the complexity of medium preparation for the cell culture and its expensiveness.

The closest analogue is the method of obtaining stem cells from human major salivary glands [WO2004074465 dated 2 Sep. 2004 “Human salivary gland-origin stem cell”]. Bioptic samples (2-4 g in weight) of human salivary glands are mechanically divided into 1-2 mm fragments, then treated with collagenase and hyaluronidase, afterwards—dispase. Monocellular suspension is washed with Williams'E medium three times and magnetically separated by CD49f marker. The sorted cells are seeded into I type collagen coated culture flasks containing Williams'E medium with addition of 20 ng/ml EGF, 10% fetal calf serum, 10⁻⁸ M insulin, 10⁻⁶ M dexamethasone, 100 U/ml penicillin, 100 μU/mI streptomycin. Under such conditions, the cell epithelial morphology destruction and proliferative activity loss are observed. In order to prevent morphological changes and enhance the passaging process, the authors suggest to: (1) maintain high concentration of cells during passage (minimum 1×10⁴ kl/cm²), (2) use conditioned medium for subculturing. Under these conditions, the cells go through at least 10 passages. Under certain conditions, salivary gland cells could be differentiated in (1) nestin and albumin-expressing, (2) insulin-expressing and (3) glucagon-expressing. This method has the following disadvantages: medium with high concentration of calcium and serum is associated with low proliferation of salivary gland epithelial cells with their further differentiation. Moreover, mesenchyme cells proliferation in this medium and their deposition in primary culture are observed. Thus, the methods used in the closest analogue for cultivation of human salivary gland cells are not optimal and do not provide a great number of passages which could maintain undifferential condition and high proliferative potential of the cells. This method does not allow to gain large mass of the human salivary gland cells.

The present invention is intended to enhance cultivation of human major salivary gland epithelial progenitor cells what allows to obtain proliferating epithelial cell culture with more than 20 passages and to provide considerable cell propagation (minimum 200 million cells from 0.5-1 cm³ bioptic sample at 2-6 passages).

### SUMMARY OF THE INVENTION

The present invention relates to culture methods of human epithelial progenitor cells.

In preferred embodiments, the method includes:

(a) obtaining human salivary gland epithelial progenitor cells from recipient organism;
(b) cell transfer into PCT Epidermal Keratinocyte Medium and cultivation in culture flasks ensuring cell adhesion at 37° C. with addition of 5% CO₂ and medium change every 2-4 days until monolayer is reached;
(c) cell passage at 1:3-1:5 dilution ratio, including cell removal from the culture flask surface using EDTA trypsin solution and transfer into the new culture flasks; (d) further cell cultivation as defined in claim (b) with in-process medium change every 2-4 days and passaging until monolayer is reached, as defined in claim (c) at a maximum dilution ratio of 1:2-1:3, where the first medium change after each passage shall be provided within 8-24 hours.
In preferred embodiments, this method applies the culture medium selected from the following group: PCT Epidermal Keratinocyte Medium CnT-07 (CELLnTEC, Switzerland); Epidermal Keratinocyte Medium CnT-09 (CELLnTEC, Switzerland); Epidermal Keratinocyte Medium CnT-57 (CELLnTEC, Switzerland); CnT-Prime, Epithelial Culture Medium CnT-PR (CELLnTEC, Switzerland); CnT-Prime 2D Diff, Epithelial Culture Medium CnT-PR-D (CELLnTEC, Switzerland); CnT-Prime Calcium Free, Epithelial Culture Medium CnT-PR-CA (CELLnTEC, Switzerland); Gingival Epithelium Progenitors, Pooled HGEPp (CELLnTEC, Switzerland); Gingival Epithelium Progenitors, Single Donor HGEPs (CELLnTEC, Switzerland).

In preferred embodiments, this method applies cell incubation according to claims (b-d) with addition of 5% O₂.

In some embodiments, immediately after obtaining the cells are incubated for 6-48 hours in DMEM/F12 1:1 medium containing at least the following additives: glutamine and fetal calf serum where the cells are incubated at 37° C. with addition of 5% CO₂. After that, the current medium shall be changed for PCT Epidermal Keratinocyte Medium.

In preferred embodiments, DMEM/F12 1:1 medium contains 1-4 mM final concentration of glutamine and 5-20% final concentration of fetal calf serum.

In preferred embodiments, all or some components selected from the following group are added in DMEM/F12 1:1 medium: insulin, transferrin, sodium selenite, epidermal growth factor (EGF).

In some embodiments, all or some components selected from the following group are added in PCT Epidermal Keratinocyte Medium: insulin, transferrin, sodium selenite, epidermal growth factor (EGF).

In some embodiments, insulin, transferrin and sodium selenite are used in the culture medium as a commercially-available ITS (Insulin-Transferrin-Selenium) additive, in other embodiments—as individual components.

The technical result of the invention is the increase in number of human salivary gland epithelial progenitor cell passages, maintaining their undifferential condition and high proliferative potential during cultivation achieved by means of optimization of cell culture conditions and application of the most suitable culture medium.

### BRIEF DESCRIPTION OF THE DRAWINGS

FIG. 1 shows graphic image of human salivary gland cells when cultivated on DMEM/F12 1:1 standard growth medium (Gibco, USA) with addition of 10% FBS (HyClone, USA), 1×ITS (Invitrogen, USA), +2 mM glutamine (Invitrogen, USA) and 10 ng/ml EGF (Sigma, USA): the cells after 2 days of cultivation (0 passage).

FIG. 2 shows graphic image of human salivary gland cells when cultivated on DMEM/F12 1:1 standard growth medium (Gibco, USA) with addition of 10% FBS (HyClone, USA), 1×ITS (Invitrogen, USA), +2 mM glutamine (Invitrogen, USA) and 10 ng/ml EGF (Sigma, USA) after the 1^{st} passage.

FIG. 3 shows graphic image of human salivary gland cells when cultivated on PCT Epidermal Keratinocyte Medium (CELLnTEC, #CnT-07, Switzerland): the cells after 16 hours of cultivation (0 passage).

FIG. 4 shows graphic image of human salivary gland cells when cultivated on PCT Epidermal Keratinocyte Medium (CELLnTEC, #CnT-07, Switzerland): the cells after 1 day of cultivation (0 passage).

FIG. 5 shows graphic image of human salivary gland cells when cultivated on PCT Epidermal Keratinocyte Medium (CELLnTEC, #CnT-07, Switzerland): the cells after 2 days of cultivation (0 passage).

FIG. 6 shows graphic image of human salivary gland cells when cultivated on PCT Epidermal Keratinocyte Medium (CELLnTEC, #CnT-07, Switzerland): the cells after 6 days of cultivation (0 passage).

FIG. 7 shows graphic image of human salivary gland cells when cultivated on PCT Epidermal Keratinocyte Medium (CELLnTEC, #CnT-07, Switzerland): the cells after 10 days of cultivation (3^{rd} passage).

FIG. 8 shows graphic image of human salivary gland cells when cultivated on PCT Epidermal Keratinocyte Medium (CELLnTEC, #CnT-07, Switzerland): the cells after 13 days of cultivation (3^{rd} passage).

FIG. 9 shows graphic image of human salivary gland cells when cultivated on PCT Epidermal Keratinocyte Medium (CELLnTEC, #CnT-07, Switzerland) with addition of 1×ITS (Invitrogen, USA), 10 ng/ml EGF (Sigma, USA): the cells after 16 hours of cultivation (0 passage).

FIG. 10 shows graphic image of human salivary gland cells when cultivated on PCT Epidermal Keratinocyte Medium (CELLnTEC, #CnT-07, Switzerland) with addition of 1×ITS (Invitrogen, USA), 10 ng/ml EGF (Sigma, USA): the cells after 1 day of cultivation (0 passage).

FIG. 11 shows graphic image of human salivary gland cells when cultivated on PCT Epidermal Keratinocyte Medium (CELLnTEC, #CnT-07, Switzerland) with addition of 1×ITS (Invitrogen, USA), 10 ng/ml EGF (Sigma, USA): the cells after 2 days of cultivation (0 passage).

FIG. 12 shows graphic image of human salivary gland cells when cultivated on PCT Epidermal Keratinocyte Medium (CELLnTEC, #CnT-07, Switzerland) with addition of 1×ITS (Invitrogen, USA), 10 ng/ml EGF (Sigma, USA): the cells after 6 days of cultivation (0 passage).

FIG. 13 shows graphic image of human salivary gland cells when cultivated on PCT Epidermal Keratinocyte Medium (CELLnTEC, #CnT-07, Switzerland) with addition of 1×ITS (Invitrogen, USA), 10 ng/ml EGF (Sigma, USA): the cells after 13 days of cultivation (4^{th} passage).

FIG. 14 shows graphic image of human salivary gland cells when cultivated on PCT Epidermal Keratinocyte Medium (CELLnTEC, #CnT-07, Switzerland) with addition of 1×ITS (Invitrogen, USA), 10 ng/ml EGF (Sigma, USA): the cells after 21 days of cultivation (4^{th} passage).

FIG. 15 shows graphic image of human salivary gland cells 5 days after isolation when cultivated on DMEM/F12 1:1 medium (Gibco, USA) with addition of 10% fetal calf serum (HyClone, USA), 2 mM glutamine (Invitrogen, USA), 1×ITS (Invitrogen, USA), 10 ng/ml EGF (Sigma, USA) (0 passage).

FIG. 16 shows graphic image of human salivary gland cells 5 days after isolation when cultivated the first 3 days on DMEM/F12 1:1 medium (Gibco, USA) with addition of 10% fetal calf serum (HyClone, USA), 2 mM glutamine (Invitrogen, USA), 1×ITS (Invitrogen, USA), 10 ng/ml EGF (Sigma, USA), and the next 2 days—on PCT Epidermal Keratinocyte Medium (CELLnTEC, #CnT-07, Switzerland) with addition of 1×ITS (Invitrogen, USA), 10 ng/ml EGF (Sigma, USA) (0 passage).

FIG. 17 shows graphic image of human salivary gland cells 10 days after isolation when cultivated the first 2 days on DMEM/F12 1:1 medium (Gibco, USA) with addition of 10% fetal calf serum (HyClone, USA), 2 mM glutamine (Invitrogen, USA), 1×ITS (Invitrogen, USA), 10 ng/ml EGF (Sigma, USA), and the next 8 days—on PCT Epidermal Keratinocyte Medium (CELLnTEC, #CnT-07, Switzerland) with addition of 1×ITS (Invitrogen, USA), 10 ng/ml EGF (Sigma, USA) (0 passage).

FIG. 18 shows graphic image of human salivary gland cells 10 days after isolation when cultivated on PCT Epidermal Keratinocyte Medium (CELLnTEC, #CnT-07, Switzerland) with addition of 1×ITS (Invitrogen, USA), 10 ng/ml EGF (Sigma, USA) (0 passage).

FIG. 19 shows graphic image of human salivary gland cells after magnetic selection by EpCAM marker: the cells 5 days after selection by EpCAM marker (2^{nd} passage).

FIG. 20 shows graphic image of human salivary gland cells after magnetic selection by EpCAM marker: selected by EpCAM cells after 7 days of cultivation after the 3^{rd} passage.

FIG. 21 shows graphic image of human salivary gland cells when continuously cultivated on PCT Epidermal Keratinocyte Medium (CELLnTEC, #CnT-07, Switzerland) with addition of 1×ITS (Invitrogen, USA), 10 ng/ml EGF (Sigma, USA): the cells in 5 days after the 4^{th} passage.

FIG. 22 shows graphic image of human salivary gland cells when continuously cultivated on PCT Epidermal Keratinocyte Medium (CELLnTEC, #CnT-07, Switzerland) with addition of 1×ITS (Invitrogen, USA), 10 ng/ml EGF (Sigma, USA): the cells in 5 days after the 20^{th} passage.

FIG. 23 shows metaphase plate karyotype of human salivary gland cells at the 5^{th} passage (G-bending).

FIG. 24 shows graphic image of human salivary gland cells after immunocytochemical staining with antibodies to cytokeratin 8 (1^{st} passage). Cell nuclei (DAPI fluorescent dye) and cytokeratin in cytoplasm (Alexa Fluor 488) are stained.

FIG. 25 shows graphic image of human salivary gland cells after immunocytochemical staining with antibodies to cytokeratin 14 (1^{st} passage). Cell nuclei (DAPI fluorescent dye) and cytokeratin in cytoplasm (Alexa Fluor 488) are stained.

FIG. 26 shows graphic image of human salivary gland cells after immunocytochemical staining with antibodies to cytokeratin 18 (1^{st} passage). Cell nuclei (DAPI fluorescent dye) and cytokeratin in cytoplasm (Alexa Fluor 488) are stained.

FIG. 27 shows graphic image of human salivary gland cells after immunocytochemical staining with antibodies to cytokeratin 19 (1^{st} passage). Cell nuclei (DAPI fluorescent dye) and cytokeratin in cytoplasm (Alexa Fluor 488) are stained.

FIG. 28 shows graphic image of human salivary gland cells after immunocytochemical staining with antibodies to GRP 49 marker of progenitor cells (1^{st} passage). Cell nuclei (DAPI fluorescent dye) and marker in cytoplasm (Alexa Fluor 488) are stained.

FIG. 29 shows graphic image of human salivary gland cells after immunocytochemical staining with antibodies to EpCAM marker of progenitor cells (1^{st} passage). Cell nuclei (DAPI fluorescent dye) and marker in cytoplasm (Alexa Fluor 488) are stained.

FIG. 30 shows graphic image of human salivary gland cells after immunocytochemical staining with antibodies to AFP marker of progenitor cells (1^{st} passage). Cell nuclei (DAPI fluorescent dye) and marker in cytoplasm (Alexa Fluor 488) are stained.

FIG. 31 shows graphic image of human salivary gland cells after immunocytochemical staining with antibodies to CD49f surface marker (1^{st} passage). Cell nuclei (DAPI fluorescent dye) and marker in cytoplasm (Alexa Fluor 488) are stained.

FIG. 32 shows graphic image of human salivary gland cells after immunocytochemical staining with antibodies to c-Met surface marker (1^{st} passage). Cell nuclei (DAPI fluorescent dye) and marker in cytoplasm (Alexa Fluor 488) are stained.

### DETAILED DESCRIPTION

As mentioned above, the present invention refers to culture methods of human salivary gland epithelial progenitor cells. The methods of this invention include obtaining human salivary gland epithelial progenitor cells from recipient organism and their cultivation providing increase in number of passages, maintaining undifferential condition and high proliferative potential of the cells during cultivation to provide cell mass gain from a small bioptic sample.

### Definitions

Various terms referred to biological molecules of the present invention are used above as well as in the description and claims.

Term “marker” or “biomarker” refers to protein or mRNA found in a certain cell type and marks it out from other cell types. So, a particular marker kit is typical for the cells referred to the present invention.

Terms “progenitor” or “undifferential” refer to stem cells determined to differentiate on various cell types (but not including terminally differentiated). Under in vitro culture conditions, progenitor cells obtain high proliferation potency and have biomarkers which allow to distinguish them from other cell types.

Progenitor epithelial cells are obtained from human major salivary glands selected by the following markers: EpCAM (NCBI Gene ID: 4072), c-Kit (NCBI Gene ID: 3815), CD49f (NCBI Gene ID: 3655), LGR5 (NCBI Gene ID: 8549).

Term “programmed differentiation” describes a set of cell formations related to a specific differential line consisting of various cell population types (for instance, stem cells, dividing cells, transitional cells), i.e. parent-progeny relationship.

Term “epithelial” describes the cells derived from epithelial tissue (epithelium)—a set of programmed differentiations of closely adjacent polarly differentiated cells (cell sheet) on basal membrane at the boundary of external or internal milieu which also form the majority of organism glands. There are two groups of epithelial tissues: surface epithelium (covering and lining) and glandular epithelium which forms the parenchyma tissue of a large portion of glands.

Term “mesenchymic” refers to mesodermal cells expressing at least the following markers: CD29, CD44, CD73, CD90, CD105. In addition, they have adipogenic, chondrogenic and osteogenic differentiation potency under certain in vitro culture conditions.

Term “immortalized” refers to cell lines prone to unlimited cell divisions under in vitro culture conditions mainly due to telomerase activation. The expression pattern of immortalized cells distinguishes from the expression pattern of unimmortalized cells.

Term “passage” or “passaging” means the adherent cell culture removal from the culture flask (protein-degrading enzyme is normally used), obtaining suspended condition and transfer of the cells to the new culture flasks with further cultivation until adherent culture formation. In terms of the present invention “zero (“0”) passage” as related to the cell culture means incubation period before the first passage, “1^{st} passage”—incubation period after the first passage and up to the second passage and so on.

Term “adherent culture” refers to the cells attached to the surface.

Term “bioptic sample” refers to biological material obtained from donor organism by means of biopsy.

Term “cultivation” means a set of methods and protocols by means of which cell viability and proliferative properties are provided under in vitro conditions.

Cells are being cultivated in culture medium. Culture medium is a nutrient solution usually containing a composition of essential amino acids, salts, vitamins, minerals, minor constituents, sugar, lipids and nucleotides. In culture medium the cells are imbued with all necessary nutritional and growth components. Culture media vary in nutrient composition, pH and osmolarity depending on cell type and cell culture density. The literature describes numerous culture media. Many of them are commercially-available, they are identified by name and, in some cases, by medium catalogue number.

Any required components can be added in the culture medium to maintain the specified cell or cell culture. For instance, growth additives or inhibitors, hormones, mammalian blood serum, containing growth factors, albumin, globulins and other components can be used.

Obtaining Epithelial Progenitor Cells from Salivary Gland

Salivary gland bioptic sample is obtained by means of biopsy or surgical operation well known in the prior art. In preferred embodiments, intravital sampling of cells and/or tissues during biopsy is applied. If the cells are intended for human use, then tissue material donor shall not have infective diseases (HIV, hepatitis B and C, syphilis) and cancers.

Right after sampling the bioptic sample shall be aseptically transferred into Petri dish containing the culture medium or isotonic solution and antibiotic. For purposes of the present invention, DMEM/F12 1:1, 199, DMEM, Eagle, Alpha-MEM, Ham, F12, IMDM, RPMI-1640 and other media can be used as well as the following isotonic solutions: phosphate-buffer saline, Hanks' solution, physiological solution, Versene solution, etc. Isotonic solution compositions are well known to researchers in the given area. Gentamycine is used as antibiotic at a final concentration of 1-100 μg/ml, for instance, 40 μg/ml, or other antibiotics: penicillin at a final concentration of 5-200 U/mI, for instance, 50 U/ml, streptomycin at a final concentration of 5-200 μg/ml, for instance, 50 μg/ml.

It is known in the art that differences in cell isolation conditions have no essential effect on further cultivation process quality.

Isotonic solutions provide the following properties: pH: from 7.3 to 7.7; osmolarity: 280+/−20 mOsm/kg; buffer capacity: minimum 1.4 ml.

All further manipulations are carried out in aseptic conditions according to GMP (Good Manufacturing Practice) requirements. Salivary gland epithelial tissue is mechanically divided by sterile tools (scalpel, forceps) into 0.5-10 mm³ fragments, more often 1-5 mm³ in size. Then tissue fragments are washed with isotonic solution twice (for instance, phosphate-buffer saline, Versene solution, physiological solution, Hanks' solution, 7.5% sodium bicarbonate, etc.), pelleted, incubated at 37° C. with addition of 0.1-10 mg/ml IV type collagenase (optimally 2 mg/ml collagenase) in DMEM/F12 1:1 medium containing 1-4 mM glutamine within 20-60 minutes. After that, cell suspension is infiltrated through nylon filter having pores 40-100 μm in diameter with further cell pelleting.

Methods of cytocentrifugation are well known in the art, for instance, cytocentrifugation per 100-400 g shall be carried out within 5-15 minutes.

Then populations enriched with epithelial progenitor cells are selected. For this purpose, various methods known in the art can be applied. For instance, the cells can be sorted out by EpCAM marker (also by c-Kit, CD49f, LGR5 markers) using magnetic or fluorescent selection.

For instance, the cells can be obtained by magnetic separation. For this purpose, cell suspension is incubated with anti-human EpCAM antibodies conjugated with magnetic particles. Manipulations shall be conducted according to the instructions of antibody producer. For instance, incubation is carried out at 4° C. (on ice) within 10-60 minutes (normally 15-40 minutes), using 0.1-10 μg of antibody per 10⁶ cells. Sorted out cells are pelleted and resuspended in culture growth medium, washed with phosphate-buffer saline and magnetically separated on columns according to the instructions of the producer.

Cultivation of Epithelial Progenitor Cells from Salivary Gland

The cells are cultivated in special sterile flasks in order to provide cell adhesion. In preferred embodiments, plastic flasks are used. As a rule, the flask internal surface is covered by bio-compatible matrix, for instance, I type collagen, IV type collagen, fibronectin or laminin in order to enhance adhesive capability.

Generally, commercially-available flasks are used for cultivation.

Obtained cells are transferred into PCT Epidermal Keratinocyte Medium and cultivated in culture flasks at 37° C. with addition of 5% CO₂, providing medium change every 2-4 days until monolayer is reached.

In some embodiments, the cells are primarily transferred into DMEM/F12 1:1 medium containing at least the following additives: glutamine and fetal calf serum. In preferred embodiments, the final concentration of glutamine in the medium is 1-4 mM, more often 1.5-3 mM, for instance, 2 mM. In preferred embodiments, the final concentration of fetal calf serum is 2-25%, more often 5-20%, normally 8-12%, for instance, 10%. In the above mentioned embodiments, the cells are cultivated in DMEM/F12 medium up to 24 hours, more often up to 18 hours, normally 10-17 hours. After that, the current culture medium is changed for PCT Epidermal Keratinocyte Medium.

In preferred embodiments, this method applies the culture medium selected from the following group: PCT Epidermal Keratinocyte Medium CnT-07 (CELLnTEC, Switzerland); Epidermal Keratinocyte Medium CnT-09 (CELLnTEC, Switzerland); Epidermal Keratinocyte Medium CnT-57 (CELLnTEC, Switzerland); CnT-Prime, Epithelial Culture Medium CnT-PR (CELLnTEC, Switzerland); CnT-Prime 2D Diff, Epithelial Culture Medium CnT-PR-D (CELLnTEC, Switzerland); CnT-Prime Calcium Free, Epithelial Culture Medium CnT-PR-CA (CELLnTEC, Switzerland); Gingival Epithelium Progenitors, Pooled HGEPp (CELLnTEC, Switzerland); Gingival Epithelium Progenitors, Single Donor HGEPs (CELLnTEC, Switzerland), for instance, PCT Epidermal Keratinocyte Medium CnT-07 can be used.

Cultivation with addition of 5% CO₂ is carried out in a special incubator maintaining constant gas composition (95% air and 5% CO₂), fixed temperature equal to 37° C. and 95-100% humidity level.

In order to reduce the death of cells while passaging, the cultivation is also carried out with addition of 5% O₂.

To increase mitotic activity and maintain undifferential condition of the cells, all or some additives selected from the following group can be used in DMEM/F12 culture medium and/or PCT Epidermal Keratinocyte Medium: insulin, transferrin, sodium selenite, epidermal growth factor. These additives increase the number of cell passages before the culture degradation and provide cell proliferation growth.

Insulin, transferrin and/or sodium selenite are added in the culture medium at a maximum final concentration of 30 μg/ml, more often 20 μg/ml maximum, normally 10 μg/mI maximum, for instance, 5-7 μg/ml. In some embodiments, insulin, transferrin and sodium selenite are used in the culture medium as a commercially-available ITS (Insulin-Transferrin-Selenium) additive, in other embodiments—as individual components.

EGF is added in the culture medium at a maximum final concentration of 200 ng/ml, more often 100 ng/ml maximum, normally 50 ng/ml maximum, for instance, 10 ng/ml.

Cell passaging is carried out to provide cell proliferation when confluent monolayer is reached. For this purpose, the culture medium is removed from the culture flask, the flask shall be washed with isotonic solution to remove residues of the culture medium. Isotonic solution is removed, then 0.05-0.25% EDTA trypsin solution in the amount of 0.5-5 ml is added. The cells with trypsin solution are incubated at 37° C., providing visual and microscopic control of cell transition into suspended condition. After all cells are removed from plastic, the cell suspension is then pelleted.

Supernatant is removed, the cells are resuspended in fresh culture medium and seeded into the new sterile culture flasks providing cell adhesion. The cells are seeded in amount of 1-50 thousands per 1 cm² of a culture flask surface area, for instance, 5 thousand cells per 1 cm² of a culture flask surface area. The cells are incubated in the culture medium under the same conditions described for the first passage.

In some embodiments, the culture flasks are washed with isotonic solution two times to provide better removal of the culture medium residues.

In preferred embodiments, in order to remove dead cells the culture medium is changed for the fresh one in 8-24 hours after passage procedure. After passaging the dead cells are not adhered and left in the culture medium. Dead cells released into the culture medium may result in spontaneous differentiation of human salivary gland cells.

During cultivation the medium is changed every 2-4 days and cell passages are provided at a maximum dilution ratio of 1:2-1:3.

Methods of the present invention ensure obtaining biomass from small bioptic samples, for instance, 0.5 cm³ in size. The obtained biomass volume is no more than 15 million cells when monolayer is reached, for instance, about 20-30 million cells. Minimum 40 million cells, for instance, about 60-90 million cells are obtained in 7-10 days after the first passage. Minimum 100 million cells, for instance, about 120-180 million cells are obtained in 7-10 days after the second passage. Minimum 200 million cells, for instance, about 240-360 million cells are obtained in 7-10 days after the third passage, that is sufficient for transplantation of cells used for cellular therapy.

The cells are counted by methods known to the persons skilled in the art. For instance, the cells can be counted by applying flow cytometry with the use of conventional antibodies to specific markers on cell surface, cell counter, in Goryaev chamber or cell sorter.

The cells obtained by methods of the present invention have potential for more than 3 passages, often more than 10 passages, normally 15 passages, usually 20 passages.

The cell culture obtained by methods of the present invention is preferably homogeneous, i.e. it contains minimum 70% epithelial progenitor cells, more often minimum 75% of epithelial progenitor cells, normally, 80% or more of epithelial progenitor cells, for instance, 90% or more of epithelial progenitor cells. For purposes of the present invention, epithelial progenitor cells can be discovered in culture by immunostaining methods or PCR to one or more markers selected from the group: EpCAM, AFP, CD49f, CK19, GRP 49, c-Met.

The cell culture obtained by methods of the present invention may contain cells expressing CD90 marker in an amount of not more than 3%, often 1.5% maximum, for instance, less than 0.5%. The cell culture obtained by methods of the present invention may contain cells expressing CD45 marker in an amount of not more than 0.5%, often 0.2% maximum, for instance, less than 0.1%.

The cell culture obtained by methods of the present invention has proliferation potency. By applying light microscopy, it is possible to visually estimate cell proliferation level by metaphase number per field of microscope and cell phenotype. Particularly, the number of cells in proliferating culture in mitosis condition is not less than 3-5%. In case of spontaneous differentiation of the culture, the cell number in mitosis condition falls below 3-5%. In addition, undifferentiated and actively proliferating cells are small in size (10-30 μm), have high nucleocytoplasmic ratio, polygonal shape, small amount of processes and form epithelial sheet in the form of cobblestone. During spontaneous differentiation the cells increase in size (more than 50 μm), often lose connectivity between themselves, obtain a great number of processes, have low nucleocytoplasmic ratio and granular cytoplasm. These cells are often multinucleated.

Proliferative rate can be also estimated by speed of reach to confluent monolayer. Cell speed of reach to confluent monolayer depends on initial dilution ratio, however, actively proliferating cells have higher speed than differentiated cells. For instance, cells reach confluent monolayer within 15 days, normally 7-10 days at a maximum dilution ratio of 1:3. Differentiated culture cells reach confluent monolayer very slow (more than 15 days) or do not reach at all.

Term “confluent monolayer” refers to the monolayer where cells cover more than 97% of the culture flask surface.

### Cell Application

The cells obtained in this invention represent actively proliferating culture of epithelial cells having potential for a great number of passages. These cells can be used as models when studying in vitro processes of mutagenesis and polyploidization, spontaneous differentiation and cell death, chromosome stability analysis in various conditions.

The cells obtained by method of the present invention can be used for investigation of biochemical and molecular mechanisms of cell proliferation and differentiation, cell cooperation, oncotransformation, cytokine expression, cytotoxicity assay of various substances, etc. As the cells obtained in the present invention are unmodified, they find use in studying of genetic expression, signaling pathways in natural biological processes. Thus, these cells can be used as models when studying mechanisms of epithelium differentiation, gland structure development and preserving the integrity of epithelial sheet, intercellular structure analysis. Co-culture allows for studying cell cooperation, mutual influence, paracrine and autocrine effects, cell mutual induction. As these cells are cultivated in large quantities, they can be used for testing of various substances in order to study their effects on biological processes (proliferation, apoptosis, differentiation) and assess their impact on cell viability, carry out medicine safety analysis and draw up cell differentiation protocols.

The cells obtained by method of the present invention can be used for autologous or allogenic transplantation for the purpose of cellular therapy. Head and neck irradiation (for instance, in beam therapy) and partial resection of salivary glands result in sialaporia caused by death (deficiency) of epithelial cells. This syndrome is followed by xerostomia. In this case, cultivated in vitro cells (both allogenic and autologous) can be transplanted to a patient in order to supplement salivary gland functions. In addition, these cells can be transplanted into other epithelial organs to stimulate their regeneration. In case of genetic deficits, cultivated in vitro allogenic cells can be transplanted to a patient in order to supplement deficient gene functions.

### EXPERIMENTAL PROCEDURE

### Example 1. Selection of Optimal Culture Medium for Human Salivary Gland Epithelial Cells

Bioptic sample was obtained from submandibular salivary gland of 37 years old male donor. The material was obtained during planned surgery operation for partial resection of salivary gland due to sialolithiasis. Minimum glandular tissue volume of bioptic sample was 2 cm³.

Bioptic sample was aseptically transferred into Petri dish containing DMEM/F12 1:1 medium (Gibco, USA) and gentamycine 40 μg/ml (PanEco, Russia). All further manipulations were carried out in sterile conditions compliant with GMP requirements.

Salivary gland epithelial tissue was separated from fatty and mesenchymal tissues using sterile tools through the binocular microscope and divided by scalpel to small fragments (about 1-5 mm³).

Then the tissue fragments were washed twice with phosphate-buffer saline, pelleted during 10 minutes at 1.5 thousand revolutions per minute, incubated at 37° C. for 30-60 minutes with addition of 2 mg/ml IV type collagenase solution (Gibco, USA) in DMEM/F12 1:1 medium (Gibco, USA) containing 2 mM glutamine (Invitrogen, USA). Tubes with salivary gland fragments were actively shaken every 10-15 minutes.

After incubation, 10 ml of DMEM/F12 1:1 medium (Gibco, USA) was added with active pipetting during 5 minutes and infiltration through nylon filter with pores 40-100 μm in diameter, then the cells were pelleted during 10 minutes at 1.5 thousand revolutions per minute.

Then the cells were magnetically separated by EpCAM marker, for which purpose the cell suspension was washed with 10 ml of phosphate-buffer saline, resuspended in 0.5 ml of phosphate-buffer saline, after that the cells were counted by use of automated cell counter (Bio-Rad, USA). Then the cells were incubated with anti-human EpCAM antibodies conjugated with magnetic particles (Miltenyi Biotec GmbH, Germany) at +4° C. during 15-40 minutes. Antibodies were added in amount of 0.1-5 μg per 10⁶ cells. After incubation the cells were washed with 10 ml of phosphate-buffer saline and magnetically separated on columns MiniMACS™ Separator (Miltenyi Biotec GmbH, Germany) according to the instructions of the producer. Sorted out cells were pelleted during 7 minutes at 200 g.

Then the cells were immersed in DMEM/F12 1:1 standard growth medium (Gibco, USA) with addition of 10% FBS (HyClone, USA), 1×ITS (Invitrogen, USA), +2 mM glutamine (Invitrogen, USA) and 10 ng/ml EGF (Sigma, USA) in amount of 5×10³ per 1 cm² and incubated in I type collagen coated culture flasks at 37° C. and 5% CO₂. The culture medium was changed every 3 days. The cells were passaged upon reaching confluent monolayer (usually in 10-15 days after cell isolation). For this purpose, the culture medium was removed, the cells were washed twice with Versene solution (PanEco, Russia) and then incubated for 5 minutes with 0.05% EDTA trypsin solution (Gibco, USA) in amount of 1 ml per 25 cm² of a culture flask surface area. Then the cells were diluted at the ratio of 1:3 and immersed in I type collagen coated new culture flasks. Cell proliferation and spontaneous differentiation were estimated every day after cell isolation: 1) by metaphase number per field of microscope; 2) by cell speed of reach to confluent monolayer; by cell phenotype.

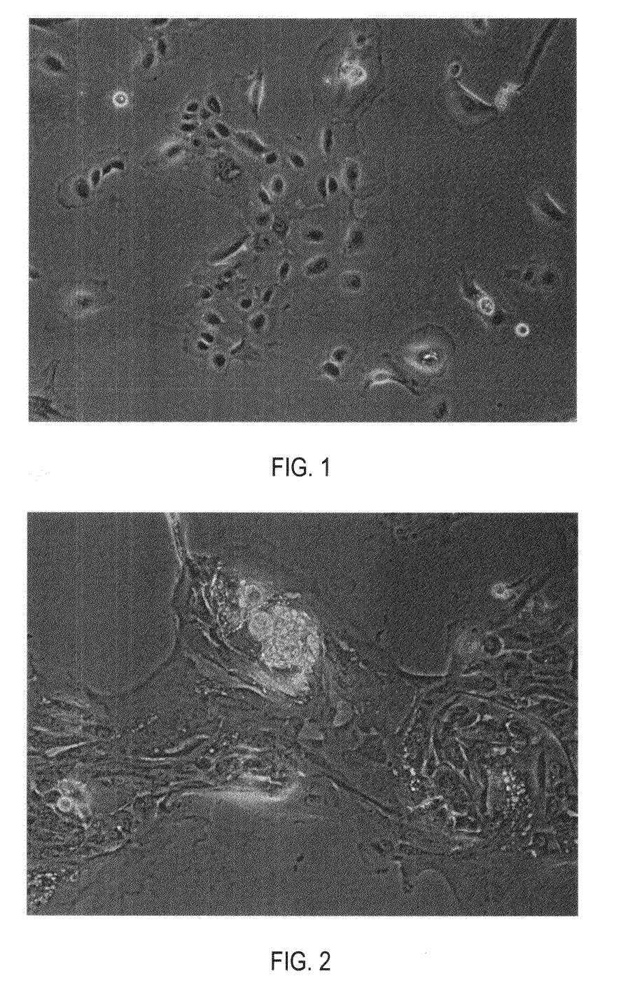

It has been found that cultivation on DMEM/F12 1:1 standard growth medium with above mentioned additives results in cell proliferation level decrease and cell differentiation increase. During the first passage the cells do not reach monolayer, increase in size, contain plenty of granules, they are polyploidized and often multinucleated (FIG. 1-2). Further gain in cell biomass appears to be impossible.

Other culture media were tested to optimize the cultivation conditions of human salivary gland epithelial progenitor cells:

PCT Epidermal Keratinocyte Medium (1×, liquid), (CELLnTEC, Switzerland), # CnT-07;

PCT Epidermal Keratinocyte Medium (1×, liquid), (CELLnTEC, Switzerland), # CnT-07 with additives: 1×ITS (Invitrogen, USA) and 10 ng/ml EGF (Sigma, USA).

The cells obtained by the above described method were seeded into I type collagen coated culture flasks (0 passage, 2^{nd} day after isolation) in amount of 5×10³ kl/cm² on standard growth medium. The next day the current medium was changed for the tested one. The day of the medium change was considered as zero experimental day (Day 0).

In all cases, the cells were cultivated as described above with tracking of cell phenotype, proliferation potency and number of passages.

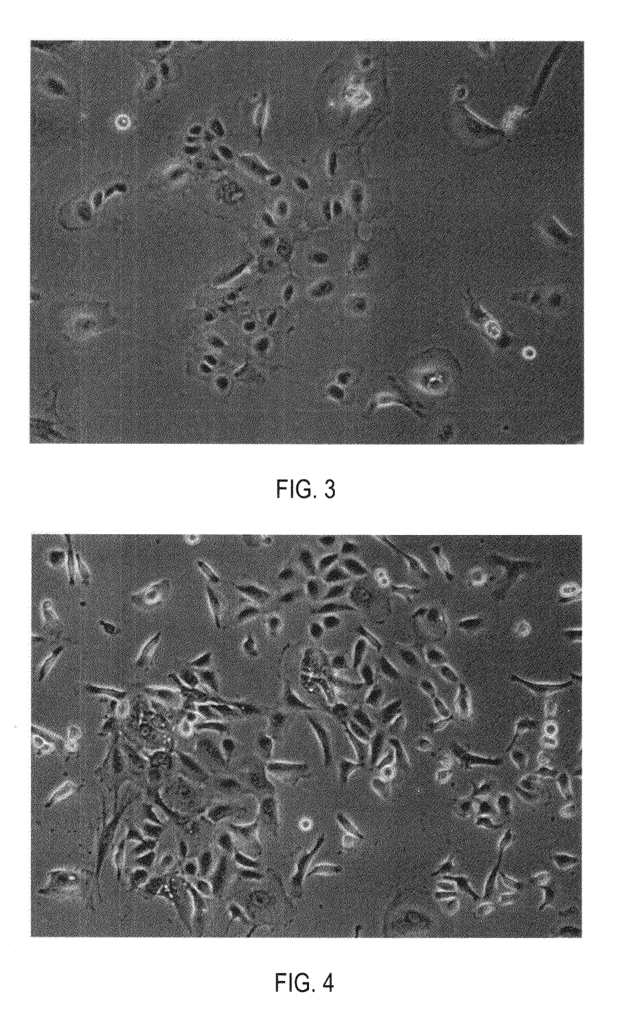

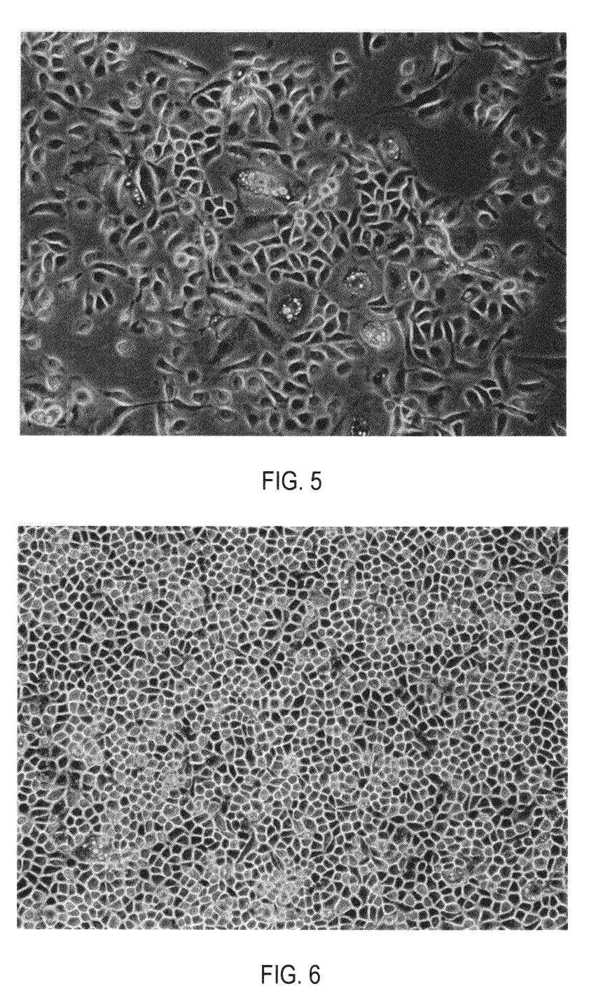

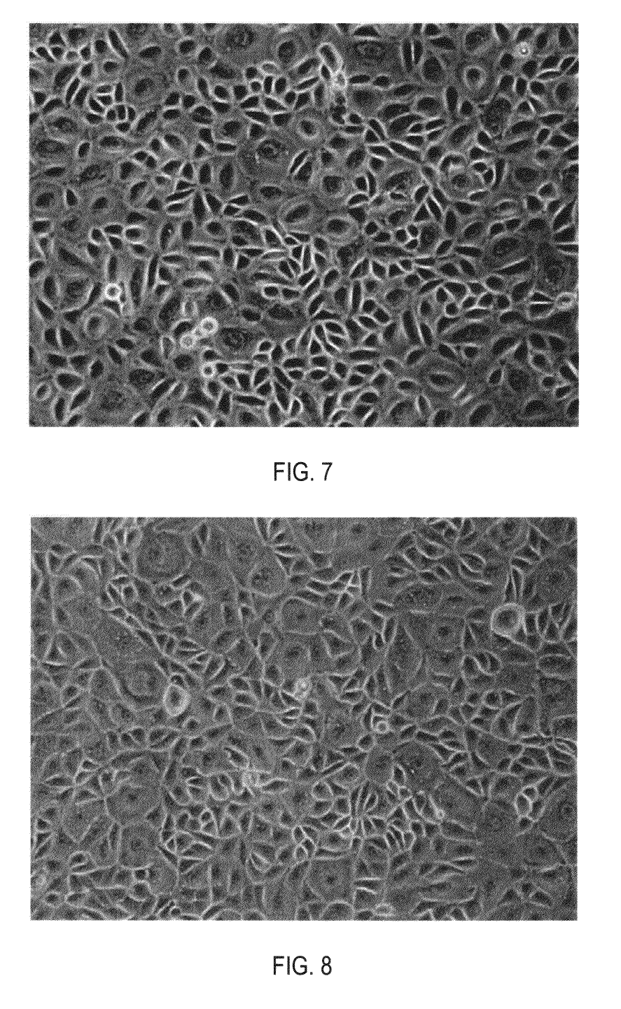

During cultivation on PCT Epidermal Keratinocyte Medium (CELLnTEC, Switzerland) the loss of cell ability to form typical epithelial sheet was observed, they obtained “migration phenotype” (cobblestone appearance) and proliferated fast (FIG. 3-8). The majority of cells maintained small size and undifferential phenotype. Particular cells were large in size, had low nucleocytoplasmic ratio and plenty of granules. The cells reached monolayer within 6 days. The current medium maintains fairly well the monolayer culture condition. Monolayer structures formation is possible when cells are continuously incubated in monolayer condition. On further passaging the cells adhered well to I type collagen coated plastic. Cell death was practically not observed.

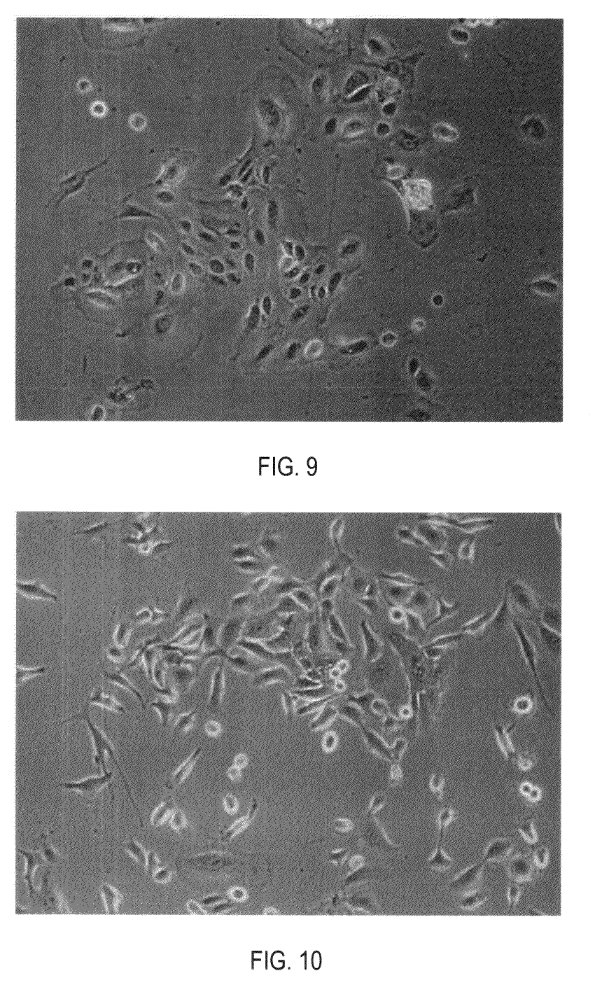

Three successful cell passages on PCT Epidermal Keratinocyte Medium (CELLnTEC, Switzerland) were observed without substantial loss of proliferation tempo (FIG. 9-10). During the third passage the clusters of small actively proliferating cells were found. There were also large cells with plenty of granules and low nucleocytoplasmic ratio.

Thus, PCT Epidermal Keratinocyte Medium (CELLnTEC, Switzerland) maintains high proliferation level and insufficiently differentiated phenotype of cultivated human salivary gland epithelial cells. The current medium is suitable for passaging human salivary gland epithelial cells.

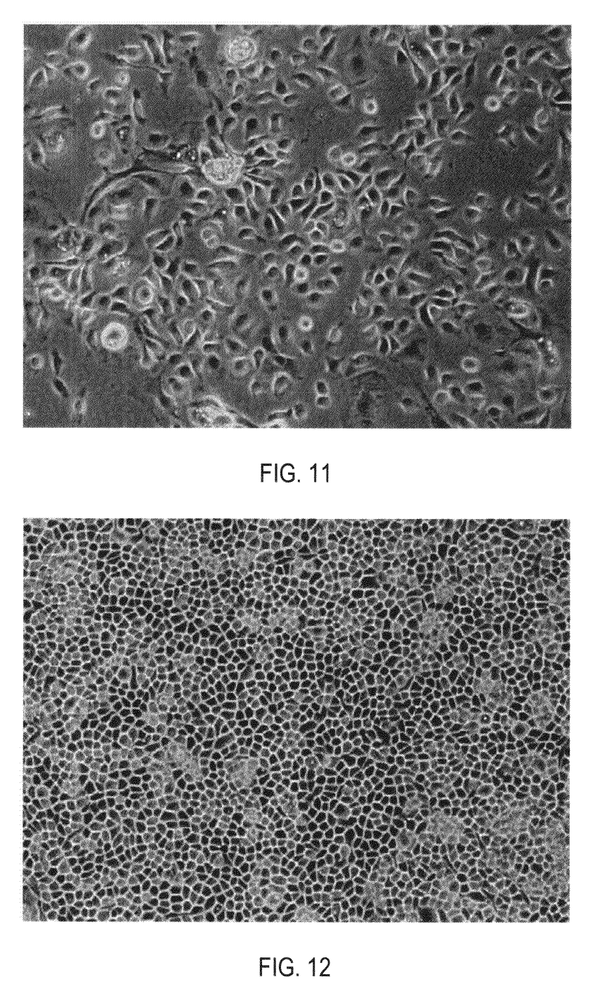

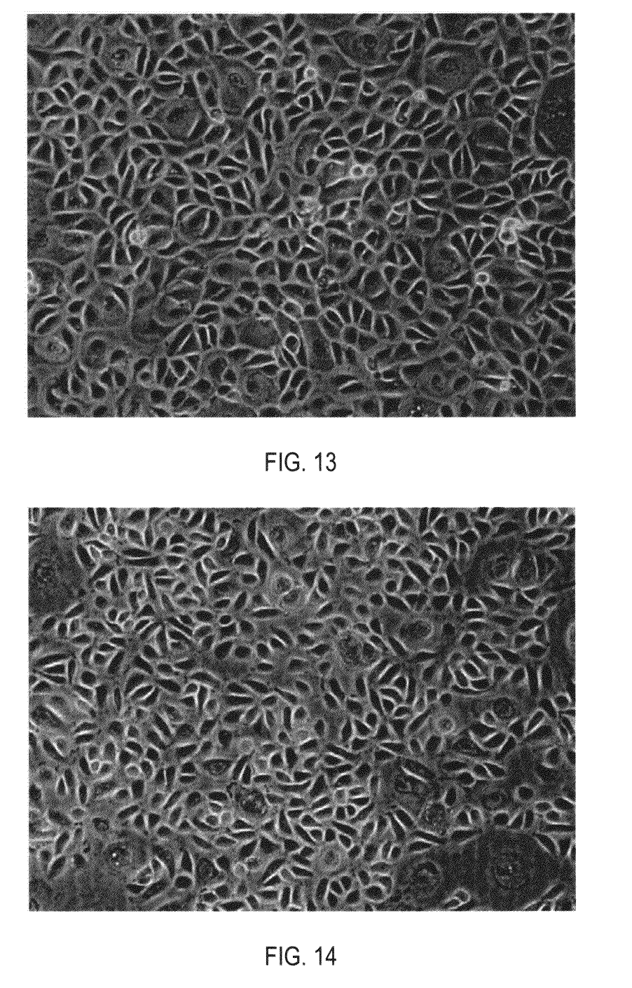

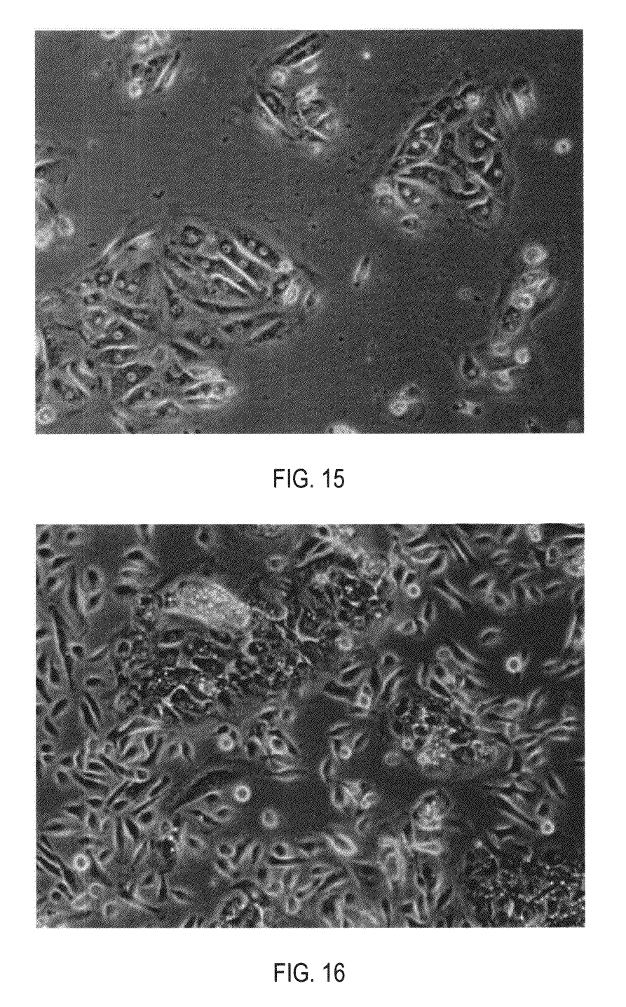

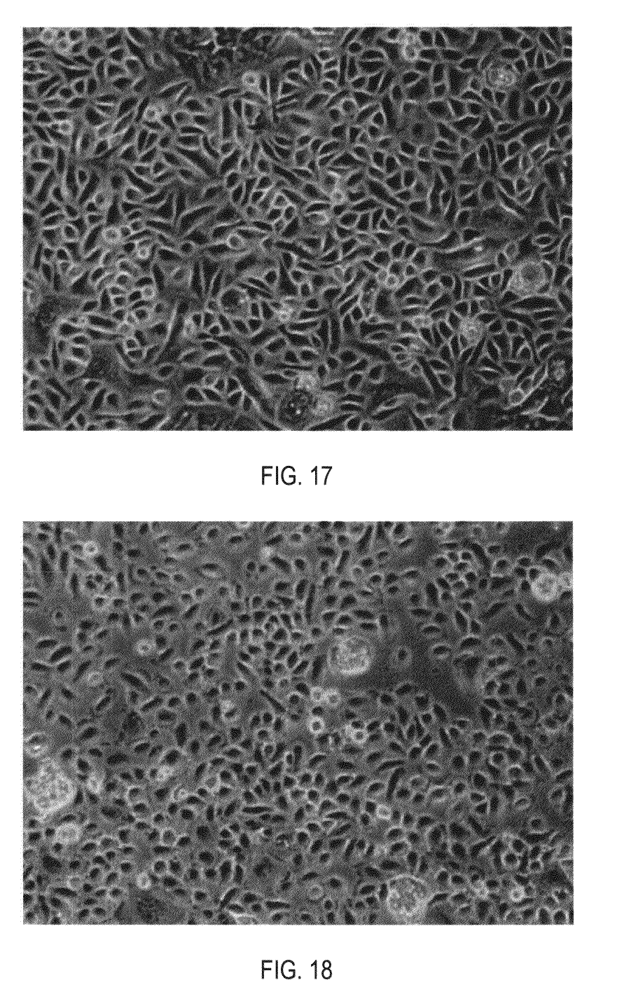

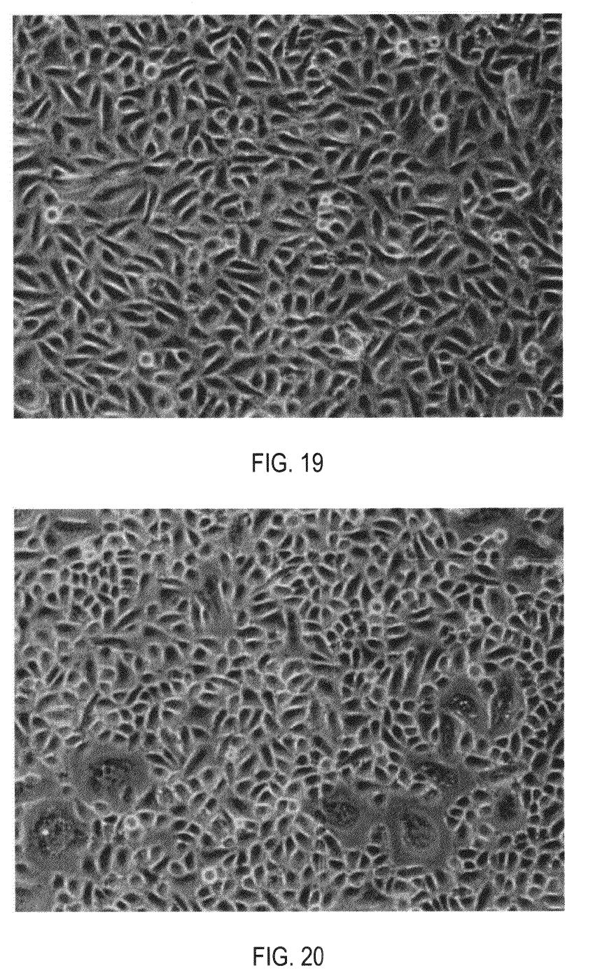

The main results when cultivating on PCT Epidermal Keratinocyte Medium CnT-07 (CELLnTEC, Switzerland) with additives 1×ITS (Invitrogen, USA) and 10 ng/ml EGF (Sigma, USA) were similar to those observed on PCT Epidermal Keratinocyte Medium CnT-07 (CELLnTEC, Switzerland) without additives (FIG. 11-16). However, application of additives resulted in cell number increase in mitosis. Along with that, the cells better cooperated during epithelial sheet formation. For this reason, the cells were cultivated on the current medium up to the 4^{th} passage (FIG. 17-18). During the 4^{th} passage, particular cells considerably increased in size (FIG. 20), still the main cell population maintained proliferation potency, epithelial phenotype and small sizes (FIG. 19). Thus, human salivary gland epithelial cells have potential for at least 4 passages without loss of proliferation ability.

Thus, PCT Epidermal Keratinocyte Medium CnT-07 (CELLnTEC, Switzerland) with additives 1×ITS (Invitrogen, USA) and 10 ng/ml EGF (Sigma, USA) maintains high proliferation level and insufficiently differentiated phenotype of cultivated human salivary gland epithelial cells. The current medium is suitable for long-term cultivation of human salivary gland epithelial progenitor cells.

### Example 2. Optimization of Culture Conditions for Human Salivary Gland Epithelial Cells

Bioptic sample was obtained from submandibular salivary gland of 50, 51 and 55 years old male donors. The material was obtained during planned surgery operation for partial resection of salivary gland due to sialolithiasis. Glandular tissue volume of bioptic sample was 2-3 cm³.

Epithelial progenitor cells were isolated as described in Example 1. Then, the cells were seeded into I type collagen coated plastic culture flasks. The cells were cultivated under standard conditions at 37° C. and 5% CO₂, varying the cultivation time in various media:

In the first embodiment, the cells were incubated in DMEM/F12 1:1 medium (Gibco, USA) with addition of 10% FBS (HyClone, USA), 1×ITS (Invitrogen, USA), 2 mM glutamine (Invitrogen, USA) and 10 ng/ml EGF (Sigma, USA) within 10 days. In the second embodiment, on first days the cells were cultivated on DMEM/F12 1:1 (Gibco, USA) with addition of 10% FBS (HyClone, USA), 1×ITS (Invitrogen, USA), 2 mM glutamine (Invitrogen, USA) and 10 ng/ml EGF (Sigma, USA), and on the third day they were transferred into PCT Epidermal Keratinocyte Medium (CELLnTEC, #CnT-07, Switzerland) with addition of 1×ITS (Invitrogen, USA), 10 ng/ml EGF (Sigma, USA). In the third embodiment, the cells were transferred into PCT Epidermal Keratinocyte Medium (CELLnTEC, #CnT-07, Switzerland) with addition of 1×ITS (Invitrogen, USA), 10 ng/ml EGF (Sigma, USA) on the second day of incubation, in the fourth embodiment—on the first day of incubation, in the fifth embodiment—the cells were immediately incubated in PCT Epidermal Keratinocyte Medium (CELLnTEC, #CnT-07, Switzerland) with addition of 1×ITS (Invitrogen, USA), 10 ng/ml EGF (Sigma, USA). Day 0 was always considered as the day of cell isolation.

In order to change the medium, it was removed from the culture flask, the cell flask was washed with phosphate-buffer saline and fresh medium was added into the culture flask.

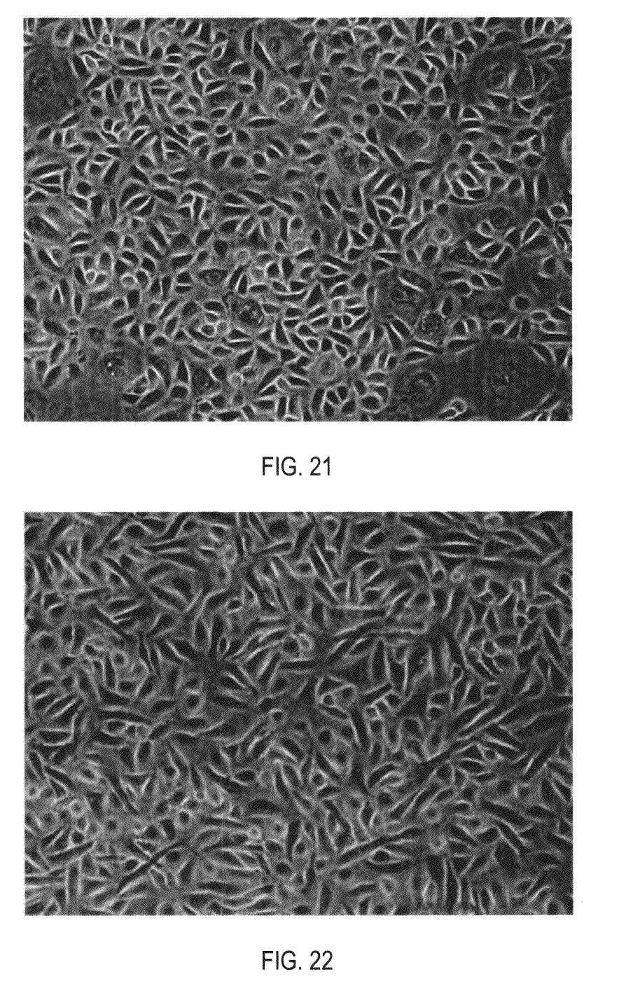

Human salivary gland cells differentiated almost immediately after transfer into DMEM/F12 1:1 medium with addition of 10% FBS, 2 RAM glutamine, 1×ITS and 10 ng/ml EGF (FIG. 21). Almost immediately after isolation (within 5 days) and sticking to substrate, the cells considerably increased in size, contained plenty of granules and proliferated poorly. After the first passage the cells did not reach monolayer as they terminally differentiated.

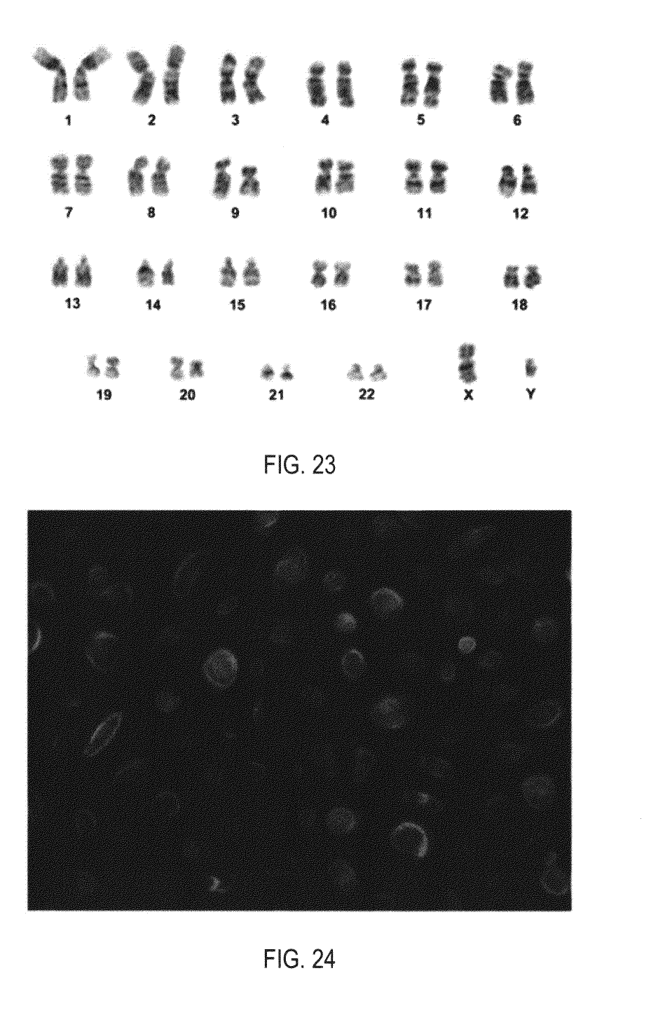

When transferred into the other medium on the 3^{rd} day after isolation (FIG. 22), the cells managed to differentiate and reached monolayer later than the cells transferred into PCT Epidermal Keratinocyte Medium (CELLnTEC, #CnT-07, Switzerland) with addition of 1×ITS (Invitrogen, USA), 10 ng/ml EGF (Sigma, USA) on the 1^{st}-2^{nd} day after isolation. When transferred into PCT Epidermal Keratinocyte Medium (CELLnTEC, #CnT-07, Switzerland) with addition of 1×ITS (Invitrogen, USA), 10 ng/ml EGF (Sigma, USA) on the 1^{st}-2^{nd} day after isolation, the cells reached dense monolayer by the 10^{th} day (FIG. 23). When seeded immediately into PCT Epidermal Keratinocyte Medium (CELLnTEC, #CnT-07, Switzerland) with addition of 1×ITS (Invitrogen, USA), 10 ng/ml EGF (Sigma, USA), the cells reached monolayer 1-2 days later (FIG. 24) due to worse sticking to plastic.

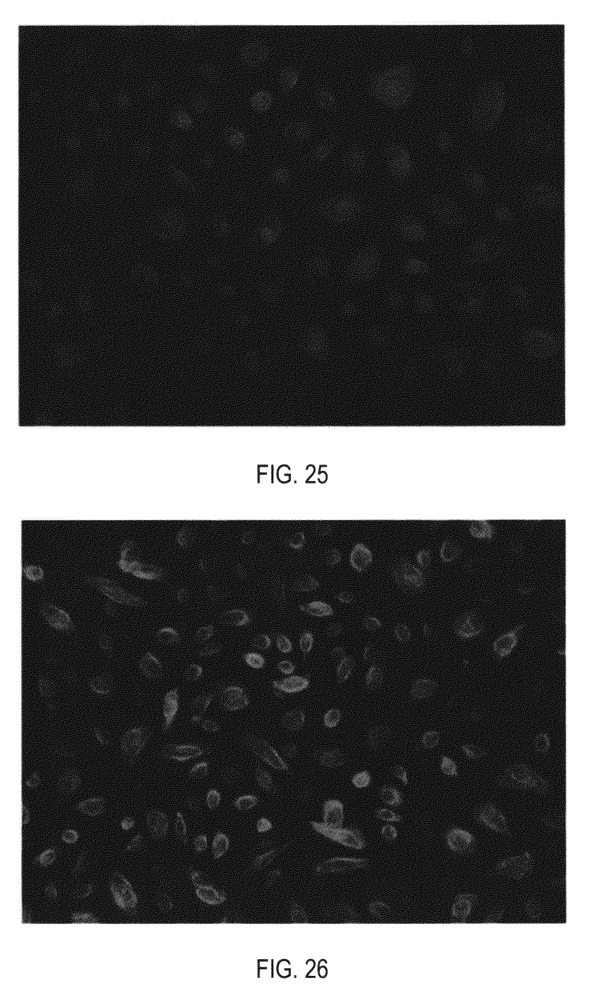

During the first passage, the cells cultivated on PCT Epidermal Keratinocyte Medium (CELLnTEC, #CnT-07, Switzerland) with addition of 1×ITS (Invitrogen, USA), 10 ng/ml EGF (Sigma, USA) were magnetically sorted by adhesive EpCAM marker of epithelial progenitor cells, as described in Example 1. It has been found that about 70% of salivary gland primary cell culture express epithelial progenitor cell EpCAM marker. After magnetic selection EpCAM expresses about 95% of salivary gland cells. After selection the culture looks homogeneous (FIG. 25) and contains small cells with high nucleocytoplasmic ratio. During the next passage there is low percentage of large cells in the culture (FIG. 26). Thus, large undifferentiated cells appear in small amounts in de novo culture. However, they do not survive after passage procedure and do not obstruct cultivation of salivary gland cells.

It has been found that when cultivated on PCT Epidermal Keratinocyte Medium (1×, liquid), (CELLnTEC, Switzerland), # CnT-07 with additives 1×ITS (Invitrogen, USA) and 10 ng/ml EGF (Sigma, USA) human salivary gland cells bear trypsinization heavily and particular cells die (sometimes up to 30-40%). This tendency was increasing in the course of passages (Table 1). To determine the amount of cells died during trypsinization, salivary gland cells were removed from the culture flask surface using trypsin solution as described above. Then the cells were resuspended in PCT Epidermal Keratinocyte Medium (1×, liquid), (CELLnTEC, Switzerland), # CnT-07 with additives 1×ITS (Invitrogen, USA) and 10 ng/ml EGF (Sigma, USA), seeded into I type collagen coated culture flasks in amount of 1.5×10⁵ per 1 cm². The percentage of dead cells was determined by counting of the cells which did not adhere to the culture medium within 15 hours after passage procedure by use of automated cell counter (BioRad, USA).

TABLE 1
Death of salivary gland cells being trypsinized during
passage on PCT Epidermal Keratinocyte Medium (1X, liquid),
(CELLnTEC, Switzerland), # CnT-07 with addition of 1xITS
(Invitrogen, USA) and 10 ng/ml EGF (Sigma, USA).

| | Salivary gland cell passage number: | | | | |
|---|---|---|---|---|---|
| | 1 | 2 | 3 | 4 | 5 |
| Percentage of | 15 ± 4 | 25 ± 5 | 30 ± 2 | 30 ± 4 | 35 ± 5 |
| dead cells, % | | | | | |
| Rate of | 7 ± 1 | 7 ± 1 | 7 ± 1 | 7 ± 1 | 7 ± 1 |
| confluent | | | | | |
| monolayer | | | | | |
| formation, days | | | | | |

It has been shown that cell death during trypsinization results from incapability of PCT Epidermal Keratinocyte Medium (1×, liquid), (CELLnTEC, Switzerland), # CnT-07 with additives 1×ITS (Invitrogen, USA) and 10 ng/ml EGF (Sigma, USA) to inhibit trypsin impeding cell adhesion. When the cells are washed from trypsin by using PCT Epidermal Keratinocyte Medium (1×, liquid), (CELLnTEC, Switzerland), Cnt-07 with addition of 5% FBS (HyClone, USA), then no massive cell death is observed. For this purpose, the cells were removed from the culture flask surface using trypsin as described above. Then cell suspension with trypsin was poured in centrifugal tubes containing double volume of PCT Epidermal Keratinocyte Medium (1×, liquid), (CELLnTEC, Switzerland), Cnt-07 with addition of 5% FBS (HyClone, USA) and pelleted for 5 minutes at 200 g. After that, supernatant was removed, the cells were resuspended in PCT Epidermal Keratinocyte Medium (1×, liquid), (CELLnTEC, Switzerland), # CnT-07 with additives 1×ITS (Invitrogen, USA) and 10 ng/ml EGF (Sigma, USA) and seeded into I type collagen coated culture flasks in amount of 1.5×10⁵ per 1 cm². The cells were cultivated in CO₂ incubator under standard conditions at 37° C. and 5% CO₂. The percentage of dead cells was determined by counting of the cells which did not adhere to the culture medium within 15 hours after passage procedure by use of automated cell counter (BioRad, USA). However, it has been shown that fetal bovine serum (FBS) intensifies cell differentiation, they stick well to plastic, gradually differentiate and discontinue proliferating (Table 2).

TABLE 2
Death of salivary gland cells being trypsinized during passage
when using PCT Epidermal Keratinocyte Medium (1X, liquid),
(CELLnTEC, Switzerland), Cnt-07 with addition of 5% FBS
(HyClone, USA) to wash from trypsin.

| | Salivary gland cell passage number: | | | | |
|---|---|---|---|---|---|
| | 1 | 2 | 3 | 4 | 5 |
| Percentage of | 3 ± 1 | 4 ± 1 | 3.5 ± 2 | 3 ± 1 | 4 ± 2 |
| dead cells, % | | | | | |
| Rate of | 7 ± 1 | 8 ± 1 | 10 ± 2 | 15 ± 1 | Do not |
| confluent | | | | | form |
| monolayer | | | | | |
| formation, days | | | | | |

It has been determined that optimized method of human salivary gland cell transplantation is washing from trypsin with centrifugation and the medium change on the next day. In this case, the rate of cell death caused by trypsinization does not exceed 30% (usually about 10%) (Table 3). For this purpose, the cells were removed from the culture flask surface using trypsin as described in Example 2. Then cell suspension with trypsin was poured in centrifugal tubes containing double volume of PCT Epidermal Keratinocyte Medium (1×, liquid), (CELLnTEC, Switzerland), Cnt-07 with additives 1×ITS (Invitrogen, USA) and 10 ng/ml EGF (Sigma, USA) and pelleted for 5 minutes at 200 g. After that, supernatant was removed, the cells were resuspended in PCT Epidermal Keratinocyte Medium (1×, liquid), (CELLnTEC, Switzerland), # CnT-07 with additives 1×ITS (Invitrogen, USA) and 10 ng/ml EGF (Sigma, USA) and seeded into I type collagen coated culture flasks in amount of 1.5×10⁵ per 1 cm². The cells were cultivated in CO₂ incubator under standard conditions at 37° C. and 5% CO₂. The percentage of dead cells was determined by counting of the cells which did not adhere to the culture medium within 15 hours after passage procedure by use of automated cell counter (BioRad, USA). The culture medium was changed for the fresh one in 16-24 hours after passaging. It provides removal of dead cells evolving decay-accelerating factors and debris which result in spontaneous cell differentiation.

TABLE 3
Death of salivary gland cells being trypsinized during
passage on PCT Epidermal Keratinocyte Medium
(1X, liquid), (CELLnTEC, Switzerland), # CnT-07
with addition of 1xITS (Invitrogen, USA) and 10 ng/ml
EGF (Sigma, USA), including washing from trypsin
and medium change in 16-24 hours.

| | Salivary gland cell passage number: | | | | |
|---|---|---|---|---|---|
| | 1 | 2 | 3 | 4 | 5 |
| Percentage of | 10 ± 2 | 11 ± 2 | 10 ± 3 | 12 ± 1 | 11 ± 1 |
| dead cells, % | | | | | |
| Rate of | 7 ± 1 | 7 ± 1 | 7 ± 1 | 7 ± 1 | 7 ± 1 |
| confluent | | | | | |
| monolayer | | | | | |
| formation, days | | | | | |

It has been also shown that when the cells are incubated in low-oxygen conditions (i.e. in CO₂ incubator at 37° C., 5% CO₂ and 5% O₂), then cell death rate during trypsinization is about 5% (Table 4). For this purpose, the cells were removed from the culture flask surface using trypsin as described above. Then cell suspension with trypsin was poured in centrifugal tubes containing double volume of PCT Epidermal Keratinocyte Medium (1×, liquid), (CELLnTEC, Switzerland), Cnt-07 with additives 1×ITS (Invitrogen, USA) and 10 ng/ml EGF (Sigma, USA) and pelleted for 5 minutes at 200 g. After that, supernatant was removed, the cells were resuspended in PCT Epidermal Keratinocyte Medium (1×, liquid), (CELLnTEC, Switzerland), # CnT-07 with additives 1×ITS (Invitrogen, USA) and 10 ng/ml EGF (Sigma, USA) and seeded into I type collagen coated culture flasks in amount of 1.5×10⁵ per 1 cm². The cells were cultivated in CO₂ incubator at 37° C., 5% CO₂ and 5% O₂. The percentage of dead cells was determined by counting of the cells which did not adhere to the culture medium within 15 hours after passage procedure by use of automated cell counter (BioRad, USA). The culture medium was changed for the fresh one in 16-24 hours after passaging. It provides removal of dead cells evolving decay-accelerating factors and debris which result in spontaneous cell differentiation.

TABLE 4
Death of salivary gland cells being trypsinized during
passage on PCT Epidermal Keratinocyte Medium
(1X, liquid), (CELLnTEC, Switzerland), # CnT-07
with addition of 1xITS (Invitrogen, USA) and 10
ng/ml EGF (Sigma, USA), including washing from
trypsin and medium change in 16-24 hours when
cultivating at 37° C., 5% CO₂ and 5% O₂.

| | Salivary gland cell passage number: | | | | |
|---|---|---|---|---|---|
| | 1 | 2 | 3 | 4 | 5 |
| Percentage of | 4 ± 1 | 5 ± 2 | 5 ± 1 | 4 ± 1 | 5 ± 1 |
| dead cells, % | | | | | |
| Rate of | 7 ± 1 | 7 ± 1 | 7 ± 1 | 7 ± 1 | 7 ± 1 |
| confluent | | | | | |
| monolayer | | | | | |
| formation, days | | | | | |

Maintaining of cell confluent monolayer condition without passaging has shown that human salivary gland cells can be remained in monolayer up to 5-7 days, otherwise the culture degrades.

The most suitable cell dilution method was determined. The best result was obtained under the following conditions: 1:3-1:5 dilution ratio is recommended during zero passage, 1:2-1:3 dilution ratio is recommended during the first passage, minimum 1:2 dilution ratio is recommended during the second and the following passages. Human salivary gland cells shall not be seeded wider than it is recommended as they differentiate fast, the culture discontinues proliferating and eventually degrades.

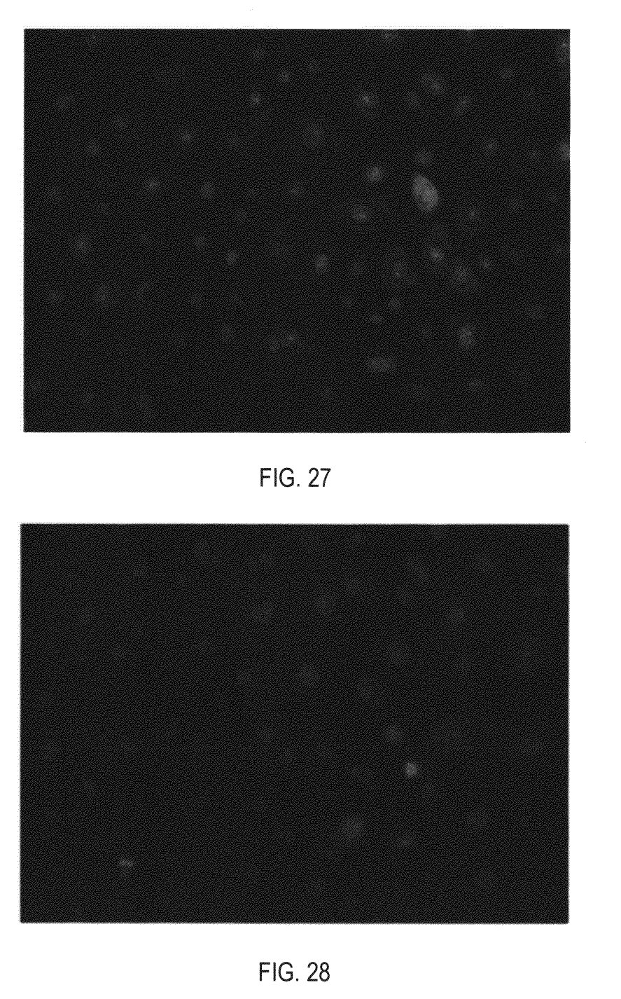

In optimum conditions, described above, the cells retained their properties and went through over 20 passages (FIG. 27-28). The culture doubling time was 40±5 hours being more or less equal for various passages. At early passages small proliferating cells and large differentiated cells were found in the culture (FIG. 27). Gradual impairment in ability to form large differentiated cells was observed, and more than 95% of the culture was homogeneous by the 10^{th} passage (FIG. 28).

In order to characterize the obtained culture the following experiments were carried out:

Cell karyotype was analyzed. Colcemid solution (Sigma, USA) at a final concentration of 0.1 mg/ml was added into the culture medium with about 70% confluent cells being further incubated at 37° C. for 40 minutes. The cells were washed with Versene solution two times, then removed from the culture flask surface using trypsin at 37° C. within 5 minutes. The cells were pelleted at 200 g for 5 minutes. Supernatant was removed, the cells were resuspended in supernatant residues and added dropwise into the tube containing 10 ml of cool fixing solution (−20° C.) composed of methanol mixture (Sigma, USA) and acetic acid (Sigma, USA) at the ratio of 3:1. The cells were incubated at −20° C. for 10 minutes. Then they were pelleted at 300 g for 10 minutes. The cells were resuspended in 300 μl of a fixing agent, pipetted on preparative glasses at a height 15 cm in amount of 40 μl per glass (glasses are prepared beforehand, they are immersed in 40% ethanol solution before specimen preparation). The specimen was set on fire until the complete burnout of a fixing agent. The specimen was incubated at 37° C. for 3 days (or at 60° C. for a night), then treated with 0.1% trypsin solution in phosphate-buffer saline at room temperature for 10-30 seconds, washed with 1% FBS solution in phosphate-buffer saline for 15 seconds and stained with Gimsa dye solution (Sigma, USA) in phosphate-buffer saline for 30 minutes. Then it was rinsed with distilled water, air dried and analyzed through the microscope using Lucia Karyo software (Laboratory Imaging s.r.o., Poland).

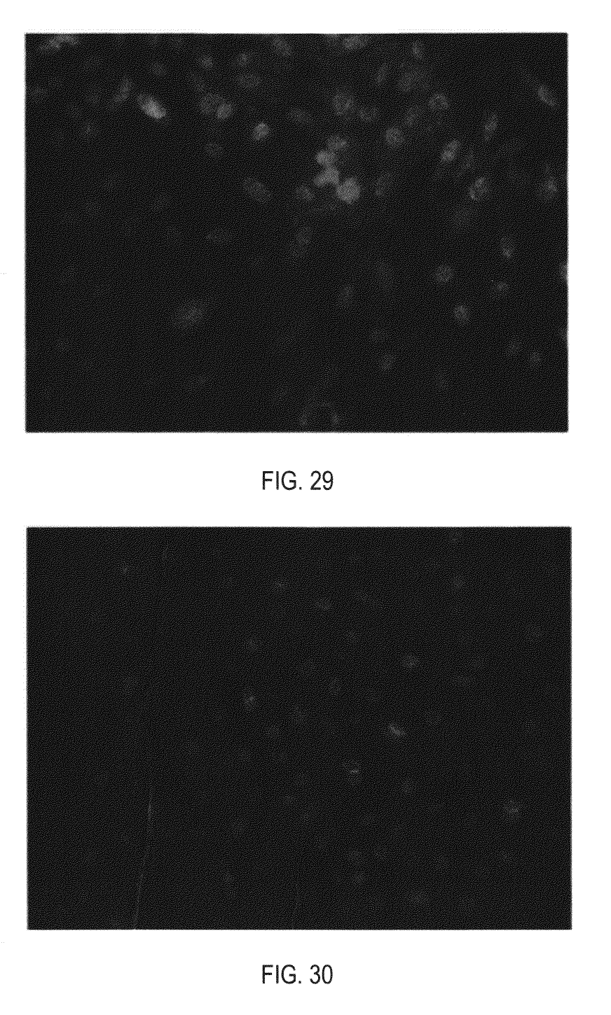

It has been shown that karyotype of salivary gland cell culture remained normal, 46XY (FIG. 29).

Cell cycle features were studied. 70% confluent cells in amount of 1-2×10⁶ were used during the analysis. The cells were removed from plastic by common techniques: washed twice with Versene solution, incubated with trypsin for about 5 minutes to obtain monocellular solution. The cells were pelleted during 5 minutes at 200 g in 10 ml of phosphate-buffer saline. The residue was resuspended and fixed by 70% cold ethanol at +4° C. for 10-30 minutes. The fixed cells were washed from a fixing solution three times by centrifugation at 200 g for 5 minutes in 10 ml of cold phosphate-buffer saline. After that, the residue was resuspended in 1 ml of phosphate-buffer saline containing 40 μg/ml propidium iodide (Calbiochem, USA) and 0.5 mg/ml ribonuclease A (Sigma, USA), incubated at 37° C. for 30 minutes. Then the cells were washed with phosphate-buffer saline, resuspended in 1 ml of phosphate-buffer saline and analyzed by flow cytometry method. Cell cycle analysis was carried out using Cell Lab Quanta SC (Beckman Coulter, USA) laser-based flow cytometry device or analog argon laser device with excitation wavelength of 488 nm and beam power of 15 mW. DNA distribution in cell cycle phases was determined by analyzing the cells stained with propidium iodide in integral fluorescent channel using 488BK, 488BP and 625BP barrier filters. Logical limitations were programmed to remove cell aggregation from analysis. The obtained histograms were mathematically processed by using MultiCycle (Phoenix Flow Systems, USA) or Flow Jo (Tree Star, Inc., USA) programs. It has been shown that about 2% of cells are in apoptosis and about 9% of cells are in mitosis during 1-3 passages, their size is about 10-13 μm; about 2% of cells are in apoptosis and about 10% of cells are in mitosis during 20^{th} passage, the cell size is about 10 μm.

The cells were analyzed by flow cytometry method at early and late passages. The cells were removed from plastic by common techniques: washed twice with Versene solution, incubated with trypsin for about 5 minutes to obtain monocellular solution. The cells were pelleted during 5 minutes at 200 g in 10 ml of phosphate-buffer saline. The residue was resuspended in phosphate-buffer saline with addition of 2% bovine serum albumin (Sigma, USA). The cells were divided into aliquots (1×10⁶ of cells per antibody plus isotype controls) and incubated with primary antibodies at the producer's recommended dilution ratio (1:500-1:1000) at room temperature in darkness for 60 minutes. Then the cells were washed with phosphate-buffer saline three times for 10 minutes at a time, pelleted at 200 g for 5 minutes, then fixed by 1% paraformaldehyde in darkness for 5 minutes. After that, the cells were washed with phosphate-buffer saline three times, resuspended in 1 ml of phosphate-buffer saline and analyzed using Cell Lab Quanta™ SC MPL (Beckman Coulter) fluorescence-based flow cytometry device. The relevant isotype control was used and at least 10 000 cells were analyzed per antibody. The flow cytometry method has shown that the obtained cells at early passages express progenitor cell markers (EpCAM, GRP 49, CK19, AFP) and epithelial cell markers (CD49f). These cells do not express hematopoietic cell marker CD45 (Table 5). At the late passages the culture becomes even more homogeneous.

TABLE 5
Human salivary gland cell analysis by flow cytometry (FCM)

| | Salivary gland cells expressing the marker, %: | |
|---|---|---|
| Marker: | 1^{st} passage | 20^{th} passage |
| AFP | 81 ± 5 | 95 ± 5 |
| CD49f | 93 ± 7 | 96 ± 3 |
| CD45 | 0.3 ± 0.2 | 0.1 ± 0.1 |
| CD90 | 1.5 ± 1 | 0.2 ± 0.1 |
| CK19 | 90 ± 4 | 96 ± 3 |
| c-Met | 94 ± 6 | 95 ± 4 |
| EpCAM | 92 ± 5 | 95 ± 4 |
| GRP 49 | 85 ± 7 | 95 ± 5 |

The cells were analyzed immunocytochemically. For immunocytochemical staining the cells were seeded into I type collagen coated flasks 48 hours before fixation. The cells were fixed by 4% paraformaldehyde at room temperature for 10 minutes, washed three times with phosphate-buffer saline with addition of 0.1% Triton X-100 and blocked by 1% bovine serum albumin (Sigma, USA) in phosphate-buffer saline at room temperature for 30 minutes. The cells together with primary antibodies were incubated in phosphate-buffer saline at 37° C. for 60 minutes at the producer's recommended dilution ratio (usually 1:200-1:500). After that, the cells were washed at 37° C. with phosphate-buffer saline three times for 10 minutes at a time, then incubated with secondary antibodies in phosphate-buffer saline (dilution at the ratio of 1:1000) at 37° C. for 40 minutes. Then the cells were washed again at 37° C. with phosphate-buffer saline three times for 10 minutes at a time, adding DAPI (Sigma, USA). The cells were analyzed using Olympus IX51 (Olympus, Japan) fluorescent microscope. The list of used antibodies is presented in Table 6.

TABLE 6
The list of antibodies used (*antibodies used for the flow cytometry)

| | | |
|---|---|---|
| Antibody | | Manufacturing company, |
| name: | Antigen: | catalogue number: |

Primary antibodies:

| | | |
|---|---|---|
| AFP* | Alpha-fetoprotein | R & D, # MAB1368 (USA) |
| CD49f* | Integrin α6 subunit | Millipore, # MAB1378 (USA) |
| CD45* | Hematopoietic cell marker | Millipore, # FCMAB126F |
| | | (USA) |
| CD90* | Thy-1 surface antigen | Millipore, # CBL1500F |
| | | (USA) |
| c-Kit | Protein kinase transmembrane | Millipore, # CBL1360 (USA) |
| | receptor, stem cell marker | |
| c-Met* | Hepatocyte growth factor | Millipore, # MAB3729 (USA) |
| | receptor | |
| EpCAM* | Adhesion molecule of | AbCam, # ab32392 |
| | epithelial cells | (Great Britain) |
| GRP 49* | Transmembrane receptor | AbCam, # ab75732 |
| | | (Great Britain) |
| KRT8 | Cytokeratin 8 | AbCam, # ab59400 |
| | | (Great Britain) |
| KRT14 | Cytokeratin 14 | Chemicon, # CBL197 (USA) |
| KRT18 | Cytokeratin 18 | Millipore, # MAB3234 (USA) |
| KRT19* | Cytokeratin 19 | AbCam, # ab15463-1 |
| | | (Great Britain) |

Secondary antibodies:

| | |
|---|---|
| Alexa Fluor ® 488 donkey anti-rabbit | Invitrogen, # A-21206 (USA) |
| IgG (H + L) | |
| Alexa Fluor ® 488 goat anti-mouse | Invitrogen, # A-11029 (USA) |
| IgG (H + L) | |
| Alexa Fluor ® 488 goat anti-rat | Invitrogen, # A-11006 (USA) |
| IgG (H + L) | |

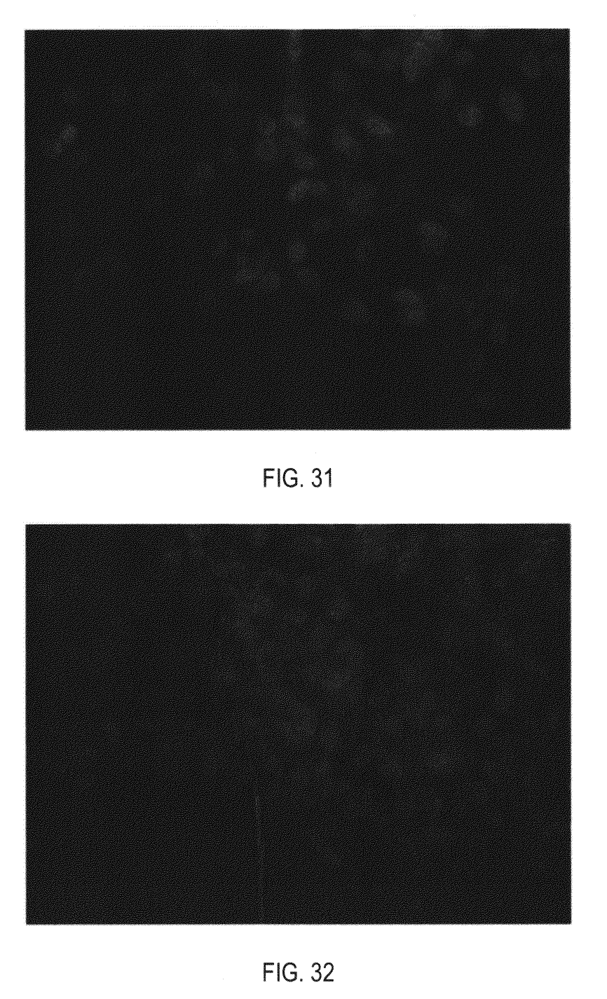

The immunocytochemistry method has shown that human salivary gland cells express cytokeratins 8, 14, 18 and 19 (FIG. 30-33) what indicates these are epithelial cells. Apart from that, they express progenitor cell markers EpCAM, GRP 49 and AFP (FIG. 34-36), as well as CD49f laminin receptor subunit and HGF c-Met receptor, what indicates these are salivary gland duct cells (FIG. 37-38).

Thus, the submitted data shows the accomplishment of the technical result—increase in number of salivary gland epithelial progenitor cell passages, maintaining undifferential condition and high proliferative potential during cultivation achieved by optimization of cell culture conditions and application of the most suitable culture medium.

Research has shown that the obtained cells are salivary gland epithelial progenitor cells, they have normal diploid karyotype and high proliferative potential under optimal conditions of isolation and cultivation.

What is claimed is:

1. A culture method of human salivary gland epithelial progenitor cells, comprising:
a) obtaining human salivary gland epithelial progenitor cells from recipient organism;
b) cell transfer into PCT Epidermal Keratinocyte Medium and cultivation in culture flasks ensuring cell adhesion at 37° C. with addition of 5% CO₂ and medium change every 2-4 days until monolayer is reached;
c) cell passage at 1:3-1:5 dilution ratio, including cell removal from the culture flask surface using EDTA trypsin solution and transfer into the new culture flasks;
d) further cell culture as defined in claim (b) with in-process medium change every 2-4 days and passages until monolayer is reached, as defined in claim (c) at a maximum dilution ratio of 1:2-1:3, where the first medium change after each passage shall be provided within 8-24 hours.

2. The method of claim 1 wherein cells are cultivated with addition of 5% of oxygen.

3. The method of claim 1 wherein immediately after obtaining of cells as defined in claim (a) they are incubated for 6-48 hours in DMEM/F12 medium containing at least the following additives: glutamine and fetal calf serum.

4. The method of claim 1 wherein PCT Epidermal Keratinocyte Medium contains components selected from the group: insulin and/or transferrin and/or sodium selenite and/or epidermal growth factor.

5. The method of claim 3 wherein DMEM/F12 medium also contains components selected from the group: insulin and/or transferrin and/or sodium selenite and/or epidermal growth factor.
